(19) United States
(12) Patent Application Publication
(10) Pub. No.: US 2005/0064038 A1
(43) Pub. Date: Mar. 24, 2005

(54) Active agent delivery systems including a single layer of a miscible polymer blend, medical devices, and methods

(76) Inventors: Thomas Q. Dinh, Minnetonka, MN (US); Randall V. Sparer, Andover, MN (US); SuPing Lyu, Maple Grove, MN (US); Kiem Dang, Blaine, MN (US); Christopher M. Hobot, Tonka Bay, MN (US)

Correspondence Address: MUETING, RAASCH & GEBHARDT, P.A., P.O. BOX 581415, MINNEAPOLIS, MN, 55458, US

(21) Appl. No.: 10/916,159

(22) Filed: Aug. 11, 2004

Related U.S. Application Data
(60) Provisional application No. 60/495,022, filed on Aug. 13, 2003.

(51) Int. Cl. A61K 9/14

(52) U.S. Cl. 424/486

(57) ABSTRACT

An active agent delivery system that includes two or more active agents in a layer of a miscible polymer blend having at least two miscible polymers; wherein delivery of at least one of the active agents occurs predominantly under permeation control; and further wherein the permeability of the active agent that is to be released faster is greater than the permeability of the other one or more active agents.

### CROSS-REFERENCE TO RELATED APPLICATIONS

The present application claims the benefit of U.S. Provisional Application No. 60/495,022, filed on 13 Aug. 2003, which is incorporated herein by reference in its entirety.

### BACKGROUND

A polymeric coating on a medical device may serve as a repository for delivery of an active agent (e.g., a therapeutic agent) to a subject. For many such applications, polymeric coatings must be as thin as possible. Polymeric materials for use in delivering an active agent may also be in various three-dimensional shapes.

Conventional active agent delivery systems suffer from limitations that include structural failure due to cracking and delamination from the device surface. Furthermore, they tend to be limited in terms of the range of active agents that can be used, the range of amounts of active agents that can be included within a delivery system, and the range of the rates at which the included active agents are delivered therefrom. This is frequently because many conventional systems include a single polymer.

Thus, there is a continuing need for active agent delivery systems with greater versatility and tunability, particularly when more than one active agent is used.

### SUMMARY OF THE INVENTION

The present invention provides active agent delivery systems that have generally good versatility and tunability in controlling the delivery of active agents. Typically, such advantages result from the use of a blend of two or more miscible polymers. These delivery systems can be incorporated into medical devices, e.g., stents, stent grafts, anastomotic connectors, if desired.

The active agent delivery systems of the present invention typically include a blend of at least two miscible polymers and two or more active agents, wherein at least one polymer (preferably one of the miscible polymers) is matched to the solubility of at least one active agent such that the delivery of at least one active agent occurs predominantly under permeation control. In this context, “predominantly” with respect to permeation control means that at least 50%, preferably at least 75%, and more preferably at least 90%, of the total active agent load is delivered by permeation control.

Permeation control is typically important in delivering an active agent from systems in which the active agent passes through a miscible polymer blend having a “critical” dimension on a micron-scale level (i.e., the diffusion net path is typically no greater than about 1000 micrometers, although for shaped objects it can be up to about 10,000 microns). Furthermore, it is generally desirable to select polymers for a particular active agent that provide desirable mechanical properties without being detrimentally affected by nonuniform incorporation of the active agent.

In a first preferred embodiment, the present invention provides an active agent delivery system (having a target diffusivity) that includes two or more active agents in a layer that includes a miscible polymer blend that includes at least two miscible polymers; wherein delivery of at least one of the active agents (and preferably all the active agents) occurs predominantly under permeation control; and further wherein the permeability of the active agent that is to be released faster is greater than the permeability of the other one or more active agents.

In a preferred embodiment, the difference between the solubility parameter of the active agent that is to be released faster and to be present in a greater amount (i.e., greater load) and the molar average solubility parameter of the at least two miscible polymers is smaller than the differences between the solubility parameter of each of the other one or more active agents and the molar average solubility parameter of the at least two miscible polymers.

In one preferred embodiment, the miscible polymer blend is hydrophilic and includes a hydrophilic polymer and a second polymer having a different swellability in water at 37° C., wherein the swellability of the miscible polymer blend controls the delivery of the active agents.

In another preferred embodiment, the miscible polymer blend includes a polyurethane and a second polymer. Preferably, the second polymer is not a hydrophobic cellulose ester.

In yet another preferred embodiment, the miscible polymer blend includes a hydrophobic cellulose derivative and a polyvinyl homopolymer or copolymer selected from the group consisting of a polyvinyl alkylate homopolymer or copolymer, a polyvinyl alkyl ether homopolymer or copolymer, a polyvinyl acetal homopolymer or copolymer, and combinations thereof.

In another preferred embodiment of the present invention, the miscible polymer blend includes copolymers of a methacrylate, a vinyl acetate, and a vinyl pyrrolidone.

In still another preferred embodiment, the miscible polymer blend includes a poly(ethylene-co-(meth)acrylate) and a second polymer. Preferably, the second polymer is not poly(ethylene vinyl acetate).

The present invention also provides medical devices (e.g., stents, stent grafts, anastomotic connectors) that include such active agent delivery systems.

The present invention also provides methods for delivering two or more active agents to a subject. In one embodiment, a method of delivery includes: providing an active agent delivery system as described above and contacting the active agent delivery system with a bodily fluid, organ, or tissue of a subject.

The present invention also provides methods for designing (and making) an active agent delivery system for delivering two or more active agent over a preselected dissolution time (t) through a preselected critical dimension (x) of a miscible polymer blend.

In one embodiment, the method includes: providing two or more active agents having a molecular weight no greater than about 1200 g/mol; selecting at least two miscible polymers to form the miscible polymer blend, wherein: the permeability of the active agent that is to be released faster is greater than the permeability of the other one or more active agents; the difference between the solubility parameter of each active agent and the molar average solubility parameter of the at least two miscible polymers is no greater than about 10 J^{1/2}/cm^{3/2}; the difference between at least one solubility parameter of each of the at least two polymers is no greater than about 5 J^{1/2}/cm^{3/2}; the difference between the solubility parameter of the active agent that is to be released faster and in a greater amount and the molar average solubility parameter of the at least two miscible polymers is smaller than the differences between the solubility parameter of each of the other one or more active agents and the molar average solubility parameter of the at least two miscible polymers; the difference between at least one Tg of each of the at least two miscible polymers is sufficient to include the target diffusivity; combining the at least two miscible polymers to form a miscible polymer blend; and combining the miscible polymer blend with the active agents to form an active agent delivery system having the preselected dissolution time through a preselected critical dimension of the miscible polymer blend, wherein delivery of at least one of the active agents occurs predominantly under permeation control.

In another embodiment, the method includes: providing two or more active agents having a molecular weight greater than about 1200 g/mol; selecting at least two miscible polymers to form the miscible polymer blend, wherein: the permeability of the active agent that is to be released faster is greater than the permeability of the other one or more active agents; the difference between the solubility parameter of each active agent and the molar average solubility parameter of the at least two miscible polymers is no greater than about 10 J^{1/2}/cm^{3/2}; the difference between at least one solubility parameter of each of the at least two miscible polymers is no greater than about 5 J^{1/2}/cm^{3/2}; the difference between the solubility parameter of the active agent that is to be released faster and in a greater amount and the molar average solubility parameter of the at least two miscible polymers is smaller than the differences between the solubility parameter of each of the other one or more active agents and the molar average solubility parameter of the at least two miscible polymers; and the difference between the swellabilities of the at least two miscible polymers is sufficient to include the target diffusivity; combining the at least two miscible polymers to form a miscible polymer blend; and combining the miscible polymer blend with the active agents to form an active agent delivery system having the preselected dissolution time through a preselected critical dimension of the miscible polymer blend, wherein delivery of at least one of the active agents occurs predominantly under permeation control.

Herein, “predominantly” in the context of permeation control means that at least 50% (preferably at least 75%, and more preferably at least 90%) of the total load of at least one active agent is delivered by permeation control. Preferably, all active agents are delivered under permeation control.

The term “permeability” is the diffusivity times solubility.

The term “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawings. FIG. 1 is idealized, not to scale, and intended to be merely illustrative and non-limiting.

FIG. 1 is a cross-section of a stent coated with a single layer of a polymer blend and therapeutic agent according to the present invention.

FIG. 2 is a graph of the release kinetics of mycophenolic acid and sulfasalazine (1:1) from a polyurethane at 30% loading.

### DETAILED DESCRIPTION OF ILLUSTRATIVE EMBODIMENTS

The present invention provides active agent delivery systems that include two or more active agents for delivery to a subject and a miscible polymer blend in a single layer. The delivery systems can include a variety of polymers as long as at least two of them are miscible as defined herein.

The active agents are incorporated within the miscible polymer blend such that at least one is delivered from the blend predominantly under permeation control. Preferably, all are delivered predominantly under permeation control. In this context, “predominantly” means that at least 50%, preferably at least 75%, and more preferably at least 90% of the total load of at least one active agent (preferably, of all the active agents) is delivered by permeation control.

In the active agent delivery systems of the present invention, an active agent is dissolutable through a miscible polymer blend. Dissolution is controlled by permeation of the active agent through the miscible polymer blend. That is, the active agent initially dissolves into the miscible polymer blend and then diffuses through the miscible polymer blend under permeation control.

When an active agent is dissoluted under permeation control, at least some solubility of the active agent in the polymer blend is required. Dispersions are acceptable as long as little or no porosity channeling occurs during dissolution of the active agent and the size of the dispersed domains is much smaller than the critical dimension of the blends, and the physical properties are generally uniform throughout the composition for desirable mechanical performance.

If the active agents exceed the solubility of the miscible polymer blend and the amount of insoluble active agent exceeds the percolation limit, then the active agent could be dissoluted predominantly through a porosity mechanism. In addition, if the largest dimension of the active agent insoluble phase (e.g., particles or aggregates of particles) is on the same order as the critical dimension of the miscible polymer blend, then the active agent could be dissoluted predominantly through a porosity mechanism. Dissolution by porosity control is typically undesirable because it does not provide effective predictability and controllability.

Because the active agent delivery systems of the present invention preferably have a critical dimension on the micron-scale level, it can be difficult to include a sufficient amount of active agent and avoid delivery by a porosity mechanism.

Thus, the active agents are preferably at or below the solubility limit of the miscible polymer blend. That is, the solubility parameters of each of the active agents and at least one polymer of the miscible polymer blend are matched to maximize the level of loading while decreasing the tendency for delivery by a porosity mechanism. Although not wishing to be bound by theory, it is believed that because of this mechanism the active agent delivery systems of the present invention have a significant level of tunability.

One can determine if there is a permeation-controlled release mechanism by examining a dissolution profile of the amount of active agent released versus time (t). For permeation-controlled release from the system, the profile is directly proportional to t^{1/2}.

The two or more active agents are selected such that the permeability of the active agent that is to be released faster is greater than the permeability of the other one or more active agents. In this context, the “permeability” of an active agent is its diffusivity times its solubility.

Preferably, the two or more active agents are selected such that the difference between the solubility parameter of the active agent that is to be released faster and to be present in a greater amount (i.e., greater load) and the molar average solubility parameter of the at least two miscible polymers is smaller than the differences between the solubility parameter of each of the other one or more active agents and the molar average solubility parameter of the at least two miscible polymers.

Miscible polymer blends are advantageous because they can provide greater versatility and tunability for a greater range of active agents than can conventional systems that include immiscible mixtures or only a single polymer, for example. That is, using two or more polymers, at least two of which are miscible, can generally provide a more versatile active agent delivery system than a delivery system with only one of the polymers. A greater range of types of active agents can typically be used. A greater range of amounts of an active agent can typically be incorporated into and delivered from (preferably, predominantly under permeation control) the delivery systems of the present invention. A greater range of delivery rates for an active agent can typically be provided by the delivery systems of the present invention. At least in part, this is because of the use of a miscible polymer blend that includes at least two miscible polymers. It should be understood that, although the description herein refers to two polymers, the invention encompasses systems that include more than two polymers, as long as a miscible polymer blend is formed that includes at least two miscible polymers.

A miscible polymer blend of the present invention has a sufficient amount of at least two miscible polymers to form a continuous portion, which helps tune the rate of release of the active agent. Such a continuous portion (i.e., continuous phase) can be identified microscopically or by selective solvent etching. Preferably, the at least two miscible polymers form at least 50 percent by volume of a miscible polymer blend.

A miscible polymer blend can also optionally include a dispersed (i.e., discontinuous) immiscible portion. If both continuous and dispersed portions are present, the active agent can be incorporated within either portion. Preferably, the active agent is loaded into the continuous portion to provide delivery of the active agent predominantly under permeation control. To load the active agent, the solubility parameters of the active agent and the portion of the miscible polymer blend a majority of the active agent is loaded into are matched (typically to within no greater than about 10 J^{1/2}/cm^{3/2}, preferably, no greater than about 5 J^{1/2}/cm^{3/2}, and more preferably, no greater than about 3 J^{1/2}/cm^{3/2}). The continuous phase controls the release of the active agent regardless of where the active agent is loaded.

A miscible polymer blend, as used herein, encompasses a number of completely miscible blends of two or more polymers as well as partially miscible blends of two or more polymers. A completely miscible polymer blend will ideally have a single glass transition temperature (Tg), preferably one in each phase (typically a hard phase and a soft phase) for segmented polymers, due to mixing at the molecular level over the entire concentration range. Partially miscible polymer blends may have multiple Tg's, which can be in one or both of the hard phase and the soft phase for segmented polymers, because mixing at the molecular level is limited to only parts of the entire concentration range. These partially miscible blends are included within the scope of the term “miscible polymer blend” as long as the absolute value of the difference in at least one Tg (Tg_{polymer 1}−Tg_{polymer 2}) for each of at least two polymers within the blend is reduced by the act of blending. Tg's can be determined by measuring the mechanical properties, thermal properties, electric properties, etc. as a function of temperature.

A miscible polymer blend can also be determined based on its optical properties. A completely miscible blend forms a stable and homogeneous domain that is transparent, whereas an immiscible blend forms a heterogeneous domain that scatters light and visually appears turbid unless the components have identical refractive indices. Additionally, a phase-separated structure of immiscible blends can be directly observed with microscopy. A simple method used in the present invention to check the miscibility involves mixing the polymers and forming a thin film of about 10 micrometers to about 50 micrometers thick. If such a film is generally as clear and transparent as the least clear and transparent film of the same thickness of the individual polymers prior to blending, then the polymers are completely miscible.

Miscibility between polymers depends on the interactions between them and their molecular structures and molecular weights. The interaction between polymers can be characterized by the so-called Flory-Huggins parameter (χ). When χ is close to zero (0) or even is negative, the polymers are very likely miscible. Theoretically, χ can be estimated from the solubility parameters of the polymers, i.e., χ is proportional to the squared difference between them. Therefore, the miscibility of polymers can be approximately predicted. For example, the closer the solubility parameters of the two polymers are the higher the possibility that the two polymers are miscible. Miscibility between polymers tends to decrease as their molecular weights increases.

Thus in addition to the experimental determinations, the miscibility between polymers can be predicted simply based on the Flory-Huggins interaction parameters, or even more simply, based the solubility parameters of the components. However, because of the molecular weight effect, close solubility parameters do not necessarily guarantee miscibility.

It should be understood that a mixture of polymers needs only to meet one of the definitions provided herein to be miscible. Furthermore, a mixture of polymers may become a miscible blend upon incorporation of an active agent.

Certain embodiments of the present invention include segmented polymers. As used herein, a “segmented polymer” is composed of multiple blocks, each of which can separate into the phase that is primarily composed of itself. As used herein, a “hard” segment or “hard” phase of a polymer is one that is either crystalline at use temperature or amorphous with a glass transition temperature above use temperature (i.e., glassy), and a “soft” segment or “soft” phase of a polymer is one that is amorphous with a glass transition temperature below use temperature (i.e., rubbery). Herein, a “segment” refers to the chemical formulation and “phase” refers to the morphology, which primarily includes the corresponding segment (e.g., hard segments form a hard phase), but can include some of the other segment (e.g., soft segments in a hard phase).

As used herein, a “hard” phase of a blend includes primarily a segmented polymer's hard segment and optionally at least part of a second polymer blended therein. Similarly, a “soft” phase of a blend includes predominantly a segmented polymer's soft segment and optionally at least part of a second polymer blended therein. Preferably, miscible blends of polymers of the present invention include blends of segmented polymers' soft segments.

When referring to the solubility parameter of a segmented polymer, “segment” is used and when referring to Tg of a segmented polymer, “phase” is used. Thus, the solubility parameter, which is typically a calculated value for segmented polymers, refers to the hard and/or soft segment of an individual polymer molecule, whereas the Tg, which is typically a measured value, refers to the hard and/or soft phase of the bulk polymer.

The types and amounts of polymers and active agents are typically selected to form a system having a preselected dissolution time through a preselected critical dimension of the miscible polymer blend. Glass transition temperatures, swellabilities, and solubility parameters of the polymers can be used in guiding one of skill in the art to select an appropriate combination of components in an active agent delivery system, whether the active agent is incorporated into the miscible polymer blend or not. Solubility parameters are generally useful for determining miscibility of the polymers and matching the solubility of the active agent to that of the miscible polymer blend. Glass transition temperatures and/or swellabilities are generally useful for tuning the dissolution time (or rate) of the active agent. These concepts are discussed in greater detail below.

Typically, the amount of active agents within an active agent delivery system of the present invention is determined by the amount to be delivered and the time period over which it is to be delivered. Other factors can also contribute to the level of active agent present, including, for example, the ability of the composition to form a uniform film on a substrate.

Preferably, each active agent is present within (i.e., incorporated within) a miscible polymer blend in an amount of at least about 0.1 weight percent (wt-%), more preferably, at least about 1 wt-%, and even more preferably, at least about 5 wt-%, based on the total weight of the miscible polymer blend and the active agents. Preferably, each active agent is present within a miscible polymer blend in an amount of no greater than about 80 wt-%, more preferably, no greater than about 50 wt-%, and most preferably, no greater than about 30 wt-%, based on the total weight of the miscible polymer blend and the active agents. Typically and preferably, the amount of each active agent will be at or below its solubility limit in the miscible polymer blend.

The active agent delivery systems of the present invention can be in the form of coatings on substrates (e.g., open or closed cell foams, woven or nonwoven materials), devices (e.g., stents, stent grafts, catheters, shunts, balloons, etc.), films (which can be free-standing as in a patch, for example), shaped objects (e.g., microspheres, beads, rods, fibers, or other shaped objects), wound packing materials, etc.

As used herein, an “active agent” is one that produces a local or systemic effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject. The term “subject” used herein is taken to include humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol and clocortolone. Preferably, the active agent is not heparin.

Certain preferred systems include an active agent selected from the group consisting of indomethacin, sulindac, diclofenal, etodolac, meclofenate, mefenamic acid, nambunetone, piroxicam, phenylgutazone, meloxicam, dexamethoasone, betamethasone, dipropionate, diflorsasone diacetate, clobetasol propionate, galobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin, curcumin, a proteasome inhibitor (e.g., bortezomib, dipeptide boronic acid, lactacystin, bisphosphonate, zolendronate, epoxomicin), antisense c-myc, celocoxib, valdecoxib, and combinations thereof. These active agents are typically selected to be the faster active agent released. Typically, it is also the first one initially released, although this is not a necessary requirement. Herein, this active agent is referred to as the first active agent.

Certain preferred systems include an active agent selected from the group consisting of podophyllotoxin, mycophenolic acid, teniposide, etoposide, trans-retinoic acids, 9-cis retinoic acid, 13-cis retinoic acid, rapamycin, a rapalog (e.g., Everolimus, ABT-578), camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof. These active agents are typically selected to be released at a slower rate than that of the first active agent, and/or after the start of release of the first active agent, for example. Generally, the concept is to release at least two active agents spread apart in time.

In certain preferred systems, one active agent is sulfasalzine, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is indomethacin, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is ascomycin, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is leflunomide, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is dexamethasone, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is piroxicam, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is beclomethasone dipropionate, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is S-nitrosoglutathione, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is rosiglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

In certain preferred systems, one active agent is troglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

In certain preferred systems, one active agent is pioglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

For preferred active agent delivery systems of the present invention, the active agent is typically matched to the solubility of the miscible portion of the polymer blend. For example, for embodiments of the invention in which the active agents are hydrophilic, preferably at least one miscible polymer of the miscible polymer blend is hydrophilic. For embodiments of the invention in which the active agents are hydrophobic, preferably at least one miscible polymer of the miscible polymer blend is hydrophobic. However, this is not necessarily required, and it may be undesirable to have a hydrophilic polymer in a delivery system for a low molecular weight hydrophilic active agent because of the potential for swelling of the polymers by water and the loss of controlled delivery of the active agent.

As used herein, in this context (in the context of the polymer of the blend), the term “hydrophilic” refers to a material that will increase in volume by more than 10% or in weight by at least 10%, whichever comes first, when swollen by water at body temperature (i.e., about 37° C.). As used herein, in this context (in the context of the polymer of the blend), the term “hydrophobic” refers to a material that will not increase in volume by more than 10% or in weight by more than 10%, whichever comes first, when swollen by water at body temperature (i.e., about 37° C.).

As used herein, in this context (in the context of the active agent), the term “hydrophilic” refers to an active agent that has a solubility in water of more than 200 micrograms per milliliter. As used herein, in this context (in the context of the active agent), the term “hydrophobic” refers to an active agent that has a solubility in water of no more than 200 micrograms per milliliter.

As the size of the active agent gets sufficiently large, diffusion through the polymer is affected. Thus, active agents can be categorized based on molecular weights and polymers can be selected depending on the range of molecular weights of the active agents.

For certain preferred active agent delivery systems of the present invention, the active agents have a molecular weight of greater than about 1200 g/mol. For certain other preferred active agent delivery systems of the present invention, the active agents have a molecular weight of no greater than (i.e., less than or equal to) about 1200 g/mol. For even more preferred embodiments, active agents of a molecular weight no greater than about 800 g/mol are desired.

Once the active agents and the format for delivery (e.g., time/rate and critical dimension) are selected, one of skill in the art can utilize the teachings of the present invention to select the appropriate combination of at least two polymers to provide an active agent delivery system.

As stated above, the types and amounts of polymers and active agents are typically selected to form a system having a preselected dissolution time (t) through a preselected critical dimension (x) of the miscible polymer blend. This involves selecting at least two polymers to provide a target diffusivity, which is directly proportional to the critical dimension squared divided by the time (x²/t), for a given active agent.

In refining the selection of the polymers for the desired active agents, the desired dissolution time (or rate), and the desired critical dimension, the parameters that can be considered when selecting the polymers for the desired active agents include glass transition temperatures of the polymers, swellabilities of the polymers, solubility parameters of the polymers, and solubility parameters of the active agents. These can be used in guiding one of skill in the art to select an appropriate combination of components in an active agent delivery system, whether the active agent is incorporated into the miscible polymer blend or not.

For enhancing the versatility of a permeation-controlled delivery system, for example, preferably the polymers are selected such that at least one of the following relationships is true: (1) the difference between the solubility parameter of the active agents and at least one solubility parameter of at least one polymer is no greater than about 10 J^{1/2}/cm^{3/2} (preferably, no greater than about 5 J^{1/2}/cm^{3/2}, and more preferably, no greater than about 3 J^{1/2}/cm^{3/2}); and (2) the difference between at least one solubility parameter of each at least two polymers is no greater than about 5 J^{1/2}/cm^{3/2} (preferably, no greater than about 3 J^{1/2}/cm^{3/2}). More preferably, both relationships are true. Most preferably, both relationships are true for all polymers of the blend.

Typically, a compound has only one solubility parameter, although certain polymers, such as segmented copolymers and block copolymers, for example, can have more than one solubility parameter. Solubility parameters can be measured or they are calculated using an average of the values calculated using the Hoy Method and the Hoftyzer-van Krevelen Method (chemical group contribution methods), as disclosed in D. W. van Krevelen, Properties of Polymers, 3^{rd} Edition, Elsevier, Amsterdam. To calculate these values, the volume of each chemical is needed, which can be calculated using the Fedors Method, disclosed in the same reference.

Solubility parameters can also be calculated with computer simulations, for example, molecular dynamics simulation and Monte Carlo simulation. Specifically, the molecular dynamics simulation can be conducted with Accelrys Materials Studio, Accelrys Inc., San Diego, Calif. The computer simulations can be used to directly calculate the Flory-Huggins parameter.

Examples of solubility parameters for various polymers and active agents is shown in Table 1.

TABLE 1

| | Solubility | | | | | |
|---|---|---|---|---|---|---|
| | parameter | | | | | |
| Polymers | (J^{1/2}/cm^{3/2}) | Source | Notes | Tg (° C.) | Notes | Source |
| polyethylene | 16.45 | 1 | | −94 | | 1 |
| polypropylene | 17.8 | 1 | | −10 | Isotactic | 1 |
| polyisobutylene | 16.3 | 1 | | −71.5 | | 1 |
| polystyrene | 18.2 | 1 | | 102.5 | Atactic | 1 |
| poly(vinyl chloride) | 20.65 | 1 | | 84 | | 1 |
| poly(vinyl bromide) | 19.4 | 1 | | | | |
| poly(vinylidene chloride) | 22.65 | 1 | | −1.5 | | 2 |
| poly(tetrafluoroethylene) | 12.7 | 1 | | 27.5 | | 1 |
| poly(chloro trifluoroethylene) | 15.45 | 1 | | 45 | | 1 |
| poly(vinyl alcohol) | 27.45 | 1 | | 85 | | 1 |
| poly(vinyl acetate) | 20.85 | 1 | | 28 | | 1 |
| poly(vinyl propionate) | 18 | 1 | | | | |
| poly(methyl acylate) | 20.6 | 1 | | 4.5 | | 1 |
| poly(ethyl acrylate) | 19 | 1 | | −24 | | 1 |
| poly(propyl acrylate) | 18.5 | 1 | | | | |
| poly(butyl acrylate) | 18.3 | 1 | | −56 | | 1 |
| poly(isobutyl acrylate) | 20.15 | 1 | | | | |
| poly(2,2,3,3,4,4,4- | 13.7 | 1 | | | | |
| heptafluorobutyl acrylate) | | | | | | |
| poly(methyl methacrylate) | 22.4 | 1 | | 105 | Atactic | 1 |
| poly(ethyl methacrylate) | 18.45 | 1 | | 65 | | 1 |
| poly(butyl methacrylate) | 18.1 | 1 | | 21 | | 1 |
| poly(isobutyl methacrylate) | 19.15 | 1 | | | | |
| poly(tert-butyl methacrylate) | 17 | 1 | | | | |
| poly(benzyl methacrylate) | 20.3 | 1 | | | | |
| poly(ethoxyethyl | 19.35 | 1 | | | | |
| methacrylate) | | | | | | |
| polyacrylonitrile | 28.55 | 1 | | 117 | Syndiotactic, | 1 |
| polymethacrylonitrile | 21.9 | 1 | | 120 | | 1 |
| poly(alpha-cyanomethyl | 29.2 | 1 | | | | |
| acrylate) | | | | | | |
| polybutadiene | 17.1 | 1 | | −50.5 | Trans 1,4- | 1 |
| | | | | | butadiene | |
| polyisoprene | 18.35 | 1 | | −59 | Trans | 1 |
| polychloroprene | 17.85 | 1 | | | | |
| polyformaldehyde | 21.7 | 1 | | −66.5 | | 1 |
| poly(tetramethylene oxide) | 17.25 | 1 | | −83.5 | | 2 |
| poly(propylene oxide) | 17.85 | 1 | | | | |
| polyepichlorohydrin | 19.2 | 1 | | | | |
| poly(ethylene sulphide) | 18.8 | 1 | | | | |
| poly(styrene sulphide) | 19 | 1 | | | | |
| poly(ethylene terephthalate) | 20.9 | 1 | | 69 | | 1 |
| poly(8-aminocaprylic acid) | 26 | 1 | | | | |
| poly(hexamethylene | 27.8 | 1 | | | | |
| adipamide) | | | | | | |
| polyurethane hard segment | 23.35 | 2 | H-vK, urethane NHCOO = NH + COO. | 10 | | RSA |
| (MDI + BDO) | | | Fedors volume | | | |
| | | | 230 cm³/mol | | | |
| poly(bisphenyl A carbonate) | 22.9 | 2 | H-vK, carbonate OCOO = COO + O; | 140 | | 1 |
| | | | Hoy OCOO = O + COO. | | | |
| | | | Fedors volume 174 cm³/mol | | | |
| cellulose acetate butyrate | 21.8 | 2 | The total numbers of acetyl, | 110 | | TSC |
| (acetyl 29.5 wt- %, butyryl | | | butyryl, and OH has to be 3 | | | |
| 17 wt- %) | | | per repeat unit. It was | | | |
| | | | estimated the wt- % of OH | | | |
| | | | was 1.1 and the molecular | | | |
| | | | weight of the repeat unit was | | | |
| | | | 303 g/mol. Fedors volume | | | |
| | | | 188 cm³/mol | | | |
| phenoxy | 23.2 | 2 | Fedors volume 201 cm³/mol | 95 | | Vendor |
| poly(vinyl pyrrolidone) | 25.1 | 2 | CON = CO + tertiary N. | 175 | | 1 |
| | | | Fedors volume 65 cm³/mol | | | |
| poly(vinyl pyrrolidone) co poly | 21.7 | 2 | CON = CO + tertiary N. | | | |
| (vinyl acetate) (1.3/1 wt) | | | Fedors volume 132 cm³/mol | | | |
| poly(ethylene oxide) | 22.15 | 2 | Fedors volume 36 cm³/mol | −47 | | 2 |
| dexamethasone | 27.25 | 2 | All rings were treated as | | | |
| | | | aliphatic. Hydroxyl groups | | | |
| | | | were not involved in | | | |
| | | | hydrogen bonding. Fedors | | | |
| | | | volume 205 cm³/mol | | | |
| Rosiglitazone maleate | 23.45 | 2 | H-vK, C5NH5 as C6H5*5/6 + tertiary | | | |
| | | | N, CONHCO as 2CO + NH; | | | |
| | | | Hoy, aromatic tertiary | | | |
| | | | N treated as aliphatic tertiary | | | |
| | | | N, CONHOC as CONH + CO. | | | |
| | | | Fedors volume 306 cm³/mol | | | |
| Source for Solubility Parameters: | | | | | | |
| 1. D. W. van Krevelen, Properties of Polymers, 3rd ed., Elsevier, 1990. Table 7.5. Data were the average if there were two values listed in the sources. | | | | | | |
| 2. Average of the calculated values based on Hoftyzer and van Kevelen's (H-vK) method (where the volumes of the chemicals were calculated based on Fedors' method) and Hoy's method. See Chapter 7, D. W. van Krevelen, Properties of Polymers, 3rd ed., Elsevier, 1990, for details of all the calculations, where Table 7.8 was for Hoftyzer | | | | | | |
| # and van Kevelen's method, Table 7.3 for Fedors' method, and Table 7.9 and 7.10 for Hoy's method. | | | | | | |

Source of Tg's (the reported value is the average if there are two values listed in the sources):
1. Table 6.6, J. M. He, W. X. Chen, and X. X. Dong, Polymer Physics, revised version, FuDan University Press, ShangHai, China, 2000. Data were the average if there were two values listed in the sources.
2. Table 6.4, D. W. van Krevelen, Properties of Polymers, 3rd ed., Elsevier, 1990. Data were the average if there were two values listed in the sources.

For delivery systems in which the active agent is hydrophobic, regardless of the molecular weight, polymers are typically selected such that the molar average solubility parameter of the miscible polymer blend is no greater than 28 J^{1/2}/cm^{3/2} (preferably, no greater than 25 J^{1/2}/cm^{3/2}). Herein “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

For example, for a hydrophobic active agent of no greater than about 1200 g/mol, such as dexamethasone, which has a solubility parameter of 27 J^{1/2}/cm^{3/2}, based on Group Contribution Methods or 21 J^{1/2}/cm^{3/2} based on Molecular Dynamics Simulations, an exemplary polymer blend includes cellulose acetate butyrate (CAB) and polyvinyl acetate (PVAC). These have solubility parameters of 22 J^{1/2}/cm^{3/2} and 21 J^{1/2}/cm^{3/2}, respectively. A suitable blend of these polymers (1:1 molar ratio is CAB to PVAC) has a molar average solubility parameter of 21.5 J^{1/2}/cm^{3/2}. This value was calculated as described herein as 22*0.5+21*0.5=21.5 (J^{1/2}/cm^{3/2}). The molecular weight of the repeat unit of CAB is estimated to be 303 g/mol based on the fact that the total number of the acetyl, butyryl, and OH groups has to be 3 per repeat unit. The molecular weight of the repeat unit of PVAC is 86 g/mol. Then the weight ratio of the CAB to PVAC=0.78/0.22 for this 1:1 molar ratio blend.

For delivery systems in which the active agent is hydrophilic, regardless of the molecular weight, polymers are typically selected such that the molar average solubility parameter of the miscible polymer blend is greater than 21 J^{1/2}/cm^{3/2} (preferably, greater than 25 J^{1/2}/cm^{3/2}).

For enhancing the tunability of permeation-controlled dissolution times (rates) for low molecular weight active agents, preferably the polymers can be selected such that the difference between at least one Tg of at least two of the polymers corresponds to a range of diffusivities that includes the target diffusivity.

Alternatively, for enhancing the tunability of permeation-controlled dissolution times (rates) for high molecular weight active agents, preferably the polymers can be selected such that the difference between the swellabilities of at least two of the polymers of the blend corresponds to a range of diffusivities that includes the target diffusivity. The target diffusivity is determined by the preselected time (t) for delivery and the preselected critical dimension (x) of the polymer composition and is directly proportional to x²/t.

The target diffusivity can be easily measured by dissolution analysis using the following equation (see, for example, Kinam Park edited, Controlled Drug Delivery: Challenges and Strategies, American Chemical Society, Washington, DC, 1997):

$$D = {( \frac{{M}_{t}}{4 {M}_{∞}} )}^{2} \cdot \frac{π {x}^{2}}{t}$$

wherein D=diffusion coefficient; Mₜ=cumulative release; M∞=total loading of active agent; x=the critical dimension (e.g., thickness of the film); and t=the dissolution time. This equation is valid during dissolution of up to 60 percent by weight of the initial load of the active agent. Also, blend samples should be in the form of a film.

Generally, at least one polymer has an active agent diffusivity higher than the target diffusivity and at least one polymer has an active agent diffusivity lower than the target diffusivity. The diffusivity of a polymer system can be easily measured by dissolution analysis, which is well known to one of skill in the art. The diffusivity of an active agent from each of the individual polymers can be determined by dissolution analysis, but can be estimated by relative Tg's or swellabilities of the major phase of each polymer.

The diffusivity can be correlated to glass transition temperatures of hydrophobic or hydrophilic polymers, which can be used to design a delivery system for low molecular weight active agents (e.g., those having a molecular weight of no greater than about 1200 g/mol). Alternatively, the diffusivity can be correlated to swellabilities of hydrophobic or hydrophilic polymers, which can be used to design a delivery system for high molecular weight polymers (e.g., those having a molecular weight of greater than about 1200 g/mol). This is advantageous because the range of miscible blends can be used to encompass very different dissolution rates for active agents of similar solubility.

The glass transition temperature of a polymer is a well-known parameter, which is typically a measured value. Exemplary values are listed in Table 1. For segmented polymers (e.g., a segmented polyurethane) the Tg refers to the particular phase of the bulk polymer. Typically, for low molecular weight active agents, by selecting relatively low and high Tg polymers that are miscible, the dissolution kinetics of the system can be tuned. This is because a small molecular weight agent (e.g., no greater than about 1200 g/mol) diffuses through a path that is directly correlated with the Tg's, i.e., the free volume of the polymer blend is a linear function of the temperature with slope being greater when the temperature is above Tg.

Preferably, a polymer having at least one relatively high Tg is combined with a polymer having at least one relatively low Tg.

For example, a miscible polymer blend for an active agent having a molecular weight of no greater than 1200 g/mol includes cellulose acetate butyrate, which has a Tg of 100-120° C., and polyvinyl acetate, which has a Tg of 20-30° C. Another example of a miscible polymer blend for an active agent having a molecular weight of no greater than 1200 g/mol includes a polyurethane with a hard phase Tg of about 10-80° C. and a polycarbonate with a Tg of about 140° C. By combining such high and low Tg polymers, the active agent delivery system can be tuned for the desired dissolution time of the active agent.

Swellabilities of polymers in water can be easily determined. It should be understood, however, that the swellability results from incorporation of water and not from an elevation in temperature. Typically, for high molecular weight active agents, by selecting relatively low and high swell polymers that are miscible, the dissolution kinetics of the system can be tuned. Swellabilities of polymers are used to design these systems because water needs to diffuse into the polymer blend to increase the free volume for active agents of relatively high molecular weight (e.g., greater than about 1200 g/mol) to diffuse out of the polymeric blend.

Preferably, a polymer having a relatively high swellability is combined with a polymer having a relatively low swellability. For example, a miscible polymer blend for an active agent having a molecular weight of greater than 1200 g/mol includes polyvinyl pyrollidone-vinyl acetate copolymer, which has a swellability of greater than 100% (i.e., it is water soluble), and poly(ether urethane), which has a swellability of 60%. By combining such high and low swell polymers, the active agent delivery system can be tuned for the desired dissolution time of the active agent.

Swellabilities of the miscible polymer blends are also used as a factor in determining the combinations of polymers for a particular active agent. For delivery systems in which the active agent has a molecular weight of greater than 1200 g/mol, whether it is hydrophilic or hydrophobic, polymers are selected such that the swellability of the blend is greater than 10% by volume. The swellability of the blend is evaluated without the active agent incorporated therein.

For a first group of active agents that are hydrophobic and have a molecular weight of no greater than about 1200 g/mol, the polymers for the miscible polymer blend are selected such that: the average molar solubility parameter of the miscible polymers of the blend is no greater than 28 J^{1/2}/cm^{3/2} (preferably, no greater than 25 J^{1/2}/cm^{3/2}); and the swellability of the blend is no greater than 10% by volume.

For a first group of active agents that have a molecular weight of greater than about 1200 g/mol, the polymers for the miscible polymer blend are selected such that: the permeability of the active agent that is to be released faster is greater than the permeability of the other one or more active agents; the difference between the solubility parameter of each active agent and the molar average solubility parameter of the at least two polymers is no greater than about 10 J^{1/2}/cm^{3/2}; the difference between at least one solubility parameter of each of the at least two polymers is no greater than about 5 J^{1/2}/cm^{3/2}; the difference between the solubility parameter of the active agent that is to be released faster and in a greater amount and the molar average solubility parameter of the at least two polymers is smaller than the differences between the solubility parameter of each of the other one or more active agents and the molar average solubility parameter of the at least two polymers; and the difference between the swellabilities of the at least two polymers is sufficient to include the target diffusivity.

For a second group of active agents that have a molecular weight of no greater than about 1200 g/mol, at least two polymers for the miscible polymer blend are selected such that: the permeability of the active agent that is to be released faster is greater than the permeability of the other one or more active agents; the difference between the solubility parameter of each active agent and the molar average solubility parameter of the at least two polymers is no greater than about 10 J^{1/2}/cm^{3/2}; the difference between at least one solubility parameter of each of the at least two polymers is no greater than about 5 J^{1/2}/cm^{3/2}; the difference between the solubility parameter of the active agent that is to be released faster and in a greater amount and the molar average solubility parameter of the at least two polymers is smaller than the differences between the solubility parameter of each of the other one or more active agents and the molar average solubility parameter of the at least two polymers; and the difference between at least one Tg of each of the at least two polymers is sufficient to include the target diffusivity.

In one preferred system of the present invention, the miscible polymer blend is hydrophilic and includes a hydrophilic polymer and a second polymer having a different swellability in water at 37° C., wherein the swellability of the miscible polymer blend controls the delivery of the active agents. The hydrophilic polymer is preferably a hydrophilic polyurethane. Alternatively, the hydrophilic polymer is selected from the group consisting of polyvinyl pyrrolidone, polyvinyl alcohol, polypropylene oxide, polyethylene oxide, polystyrene sulfonate, polysaccharide, and combinations thereof. The second polymer can be hydrophilic (e.g., a hydrophilic polyurethane that includes soft segments of polyethylene oxide units) or hydrophobic. In a particularly preferred embodiment, the miscible polymer blend includes a polyvinyl pyrollidone-co-vinyl acetate copolymer and a poly(ether urethane).

In another preferred embodiment of the present invention, the miscible polymer blend includes a polyurethane and a second polymer, which preferably has at least one Tg equal to or higher than all Tg's of the polyurethane. Examples of suitable second polymers include a polycarbonate, a polysulfone, a polyurethane, a polyphenylene oxide, a polyimide, a polyamide, a polyester, a polyether, a polyketone, a polyepoxide, a styrene-acrylonitrile copolymer, or combinations thereof. Preferably, the second polymer is not a hydrophobic cellulose ester. Preferably, the second polymer is a polycarbonate. For preferred embodiments, the active agent is not heparin. For certain embodiments, the polyurethane has a Shore durometer hardness of about 70D to about 80D for one embodiment and for certain other embodiments, the polyurethane has a Shore durometer hardness of about 80D to about 90D for another embodiment. The polyurethane can be a poly(carbonate urethane) or a poly(ether urethane).

For certain embodiments of the system in which the miscible polymer blend includes a polyurethane and a second polymer (which preferably has at least one Tg equal to or higher than all Tg's of the polyurethane), each active agent has a solubility parameter, the polyurethane has a soft segment solubility parameter and a hard segment solubility parameter, and the second polymer has at least one solubility parameter. Furthermore, at least one of the following relationships is true: the difference between the solubility parameter of each active agent and the solubility parameter of the polyurethane hard segment is no greater than about 10 J^{1/2}/cm^{3/2}; the difference between the solubility parameter of each active agent and the solubility parameter of the polyurethane soft segment is no greater than about 10 J^{1/2}/cm^{3/2}; and the difference between the solubility parameter of each active agent and at least one solubility parameter of the second polymer is no greater than about 10 J^{1/2}/cm^{3/2}.

For certain embodiments of the system in which the miscible polymer blend includes a polyurethane and a second polymer (which preferably has at least one Tg equal to or higher than all Tg's of the polyurethane), the polyurethane has a soft segment solubility parameter and a hard segment solubility parameter, and the second polymer has at least one solubility parameter. Furthermore, and at least one of the following relationships is true: the difference between the solubility parameter of the polyurethane hard segment and at least one solubility parameter of the second polymer is no greater than about 5 J^{1/2}/cm^{3/2}; and the difference between the solubility parameter of the polyurethane soft segment and at least one solubility parameter of the second polymer is no greater than about 5 J^{1/2}/cm^{3/2}.

In another preferred embodiment of the present invention, the miscible polymer blend includes a hydrophobic cellulose derivative and a polyvinyl homopolymer or copolymer selected from the group consisting of a polyvinyl alkylate homopolymer or copolymer, a polyvinyl alkyl ether homopolymer or copolymer, a polyvinyl acetal homopolymer or copolymer, and combinations thereof. Preferably, the polyvinyl homopolymer or copolymer is a polyvinyl alkylate homopolymer or copolymer (e.g., a homopolymer or copolymer of polyvinyl acetate, polyvinyl propionate, or polyvinyl butyrate), and more preferably, a polyvinyl acetate homopolymer or copolymer. Examples of suitable hydrophobic cellulose derivatives include methyl cellulose, ethyl cellulose, hydroxy propyl cellulose, cellulose acetate, cellulose propionate, cellulose butyrate, cellulose nitrate, or combinations thereof.

For certain embodiments wherein the miscible polymer blend includes a hydrophobic cellulose derivative and a polyvinyl homopolymer or copolymer, each of the active agents, the hydrophobic cellulose derivative, and the polyvinyl homopolymer or copolymer has a solubility parameter; and at least one of the following relationships is true: the difference between the solubility parameter of each active agent and the solubility parameter of the hydrophobic cellulose derivative is no greater than about 10 J^{1/2}/cm^{3/2}; and the difference between the solubility parameter of each active agent and at least one solubility parameter of the polyvinyl homopolymer or copolymer is no greater than about 10 J^{1/2}/cm^{3/2}. Preferably, each active agent has a solubility parameter within at least about 10 J^{1/2}/cm^{3/2} of the solubility parameters of each of cellulose acetate butyrate and polyvinyl acetate.

For certain embodiments wherein the miscible polymer blend includes a hydrophobic cellulose derivative and a polyvinyl homopolymer or copolymer, each of the hydrophobic cellulose derivative and the polyvinyl homopolymer or copolymer has a solubility parameter; and the difference between the solubility parameter of the hydrophobic cellulose derivative and at least one solubility parameter of the polyvinyl homopolymer or copolymer is no greater than about 5 J^{1/2}/cm^{3/2}.

In another preferred embodiment of the present invention, the miscible polymer blend includes a poly(ethylene-co-(meth)acrylate) and a second polymer, which is preferably not poly(ethylene vinyl acetate). Preferably, each of the active agents, the poly(ethylene-co-(meth)acrylate) and the second polymer has a solubility parameter; and at least one of the following relationships is true: the difference between the solubility parameter of each active agent and the solubility parameter of the poly(ethylene-co-(meth)acrylate) is no greater than about 10 J^{1/2}/cm^{3/2}; and the difference between the solubility parameter of each active agent and at least one solubility parameter of the second polymer is no greater than about 10 J^{1/2}/cm^{3/2}. Preferably, each of the poly(ethylene-co-(meth)acrylate) and the second polymer has a solubility parameter; and the difference between the solubility parameter of the poly(ethylene-co-(meth)acrylate) and at least one solubility parameter of the second polymer is no greater than about 5 J^{1/2}/cm^{3/2}. The second polymer can be a polyvinyl alkylate homopolymer or copolymer, a polyalkyl and/or aryl methacrylate or acrylate or copolymer, or a polyvinyl acetal or copolymer.

In another preferred embodiment of the present invention, the miscible polymer blend includes a copolymer of (methacrylates, vinyl acetate, and vinyl pyrrolidone) and a second polymer, which is preferably another copolymer of (methacrylates, vinyl acetate and vinyl pyrrolidone) with different compositions of methacrylates, vinyl acetate and vinyl pyrrolidone from the first copolymer. Preferably, each of the active agents, the first copolymer and the second copolymer of methacrylates, vinyl acetate and vinyl pyrrolidone has a solubility parameter; and at least one of the following relationships is true: the difference between the solubility parameter of each active agent and the solubility parameter of the first copolymer of (methacrylates, vinyl acetate and vinyl pyrrolidone) is no greater than about 10 J^{1/2}/cm^{3/2}; and the difference between the solubility parameter of each active agent and the solubility parameter of the second copolymer (methacrylates, vinyl acetate and vinyl pyrrolidone) is no greater than about 10 J^{1/2}/cm^{3/2}. Preferably, each of the first copolymer of (methacrylates, vinyl acetate and vinyl pyrrolidone) and the second copolymer of (methacrylates, vinyl acetate and vinyl pyrrolidone) has a solubility parameter; and the difference between the solubility parameter of the first copolymer of (methacrylates, vinyl acetate and vinyl pyrrolidone) and the solubility parameter of the second copolymer of (methacrylates, vinyl acetate and vinyl pyrrolidone) is no greater than about 5 J^{1/2}/cm^{3/2}.

The polymers in the miscible polymer blends can be crosslinked or not. Similarly, the blended polymers can be crosslinked or not. Such crosslinking can be carried out by one of skill in the art after blending using standard techniques.

In the active agent systems of the present invention, the active agents pass through a miscible polymer blend having a “critical” dimension. This critical dimension is along the net diffusion path of the active agents and is preferably no greater than about 1000 micrometers (i.e., microns), although for shaped objects it can be up to about 10,000 microns.

For embodiments in which the miscible polymer blends form coatings or free-standing films (both generically referred to herein as “films”), the critical dimension is the thickness of the film and is preferably no greater than about 1000 microns, more preferably no greater than about 500 microns, and most preferably no greater than about 100 microns. A film can be as thin as desired (e.g., 1 nanometer), but are preferably no thinner than about 10 nanometers, more preferably no thinner than about 100 nanometers. Generally, the minimum film thickness is determined by the volume that is needed to hold the required doses of active agents and is typically only limited by the process used to form the materials. For all embodiments herein, the thickness of the film does not have to be constant or uniform. Furthermore, the thickness of the film can be used to tune the duration of time over which the active agent is released.

For embodiments in which the miscible polymer blends form shaped objects (e.g., microspheres, beads, rods, fibers, or other shaped objects), the critical dimension of the object (e.g., the diameter of a microsphere or rod) is preferably no greater than about 10,000 microns, more preferably no greater than about 1000 microns, even more preferably no greater than about 500 microns, and most preferably no greater than about 100 microns. The objects can be as small as desired (e.g., 10 nanometers for the critical dimension). Preferably, the critical dimension is no less than about 100 microns, and more preferably no less than about 500 nanometers.

In one embodiment, the present invention provides a medical device characterized by a substrate surface overlayed with a polymeric top coat layer that includes a miscible polymer blend, preferably with a polymeric undercoat (primer) layer. When the device is in use, the miscible polymer blend is in contact with a bodily fluid, organ, or tissue of a subject.

The invention is not limited by the nature of the medical device; rather, any medical device can include the polymeric coating layer that includes the miscible polymer blend. Thus, as used herein, the term “medical device” refers generally to any device that has surfaces that can, in the ordinary course of their use and operation, contact bodily tissue, organs or fluids such as blood. Examples of medical devices include, without limitation, stents, stent grafts, anastomotic connectors, leads, needles, guide wires, catheters, sensors, surgical instruments, angioplasty balloons, wound drains, shunts, tubing, urethral inserts, pellets, implants, pumps, vascular grafts, valves, pacemakers, and the like. A medical device can be an extracorporeal device, such as a device used during surgery, which includes, for example, a blood oxygenator, blood pump, blood sensor, or tubing used to carry blood, and the like, which contact blood which is then returned to the subject. A medical device can likewise be an implantable device such as a vascular graft, stent, stent graft, anastomotic connector, electrical stimulation lead, heart valve, orthopedic device, catheter, shunt, sensor, replacement device for nucleus pulposus, cochlear or middle ear implant, intraocular lens, and the like. Implantable devices include transcutaneous devices such as drug injection ports and the like.

In general, preferred materials used to fabricate the medical device of the invention are biomaterials. A “biomaterial” is a material that is intended for implantation in the human body and/or contact with bodily fluids, tissues, organs and the like, and that has the physical properties such as strength, elasticity, permeability and flexibility required to function for the intended purpose. For implantable devices in particular, the materials used are preferably biocompatible materials, i.e., materials that are not overly toxic to cells or tissue and do not cause undue harm to the body.

The invention is not limited by the nature of the substrate surface for embodiments in which the miscible polymer blends form polymeric coatings. For example, the substrate surface can be composed of ceramic, glass, metal, polymer, or any combination thereof. In embodiments having a metal substrate surface, the metal is typically iron, nickel, gold, cobalt, copper, chrome, molybdenum, titanium, tantalum, aluminum, silver, platinum, carbon, and alloys thereof. A preferred metal is stainless steel, a nickel titanium alloy, such as NITINOL, or a cobalt chrome alloy, such as NP35N.

A polymeric coating that includes a miscible polymer blend can adhere to a substrate surface by either covalent or non-covalent interactions. Non-covalent interactions include ionic interactions, hydrogen bonding, dipole interactions, hydrophobic interactions and van der Waals interactions, for example.

Preferably, the substrate surface is not activated or functionalized prior to application of the miscible polymer blend coating, although in some embodiments pretreatment of the substrate surface may be desirable to promote adhesion. For example, a polymeric undercoat layer (i.e., primer) can be used to enhance adhesion of the polymeric coating to the substrate surface. Suitable polymeric undercoat layers are disclosed in Applicants' Assignee's copending U.S. Provisional Application Serial No. 60/403,479, filed on Aug. 13, 2002; U.S. patent application Ser. No. 10/640,701, filed Aug. 13, 2003; and PCT International Patent Application No. PCT/US 03/25463, filed Aug. 13, 2003 (published as WO 2004/014453A1 on Feb. 19, 2004), all of which are entitled MEDICAL DEVICE EXHIBITING IMPROVED ADHESION BETWEEN POLYMERIC COATING AND SUBSTRATE. A particularly preferred undercoat layer disclosed therein consists essentially of a polyurethane material. Such a preferred undercoat layer includes a polymer blend that contains polymers other than polyurethane but only in amounts so small that they do not appreciably affect the durometer, durability, adhesive properties, structural integrity and elasticity of the undercoat layer compared to an undercoat layer that is exclusively polyurethane.

When a stent or other vascular prosthesis is implanted into a subject, restenosis is often observed during the period beginning shortly after injury to about four to six months later. Thus, for embodiments of the invention that include stents, the generalized dissolution rates contemplated are such that the active agents should ideally start to be released immediately after the prosthesis is secured to the lumen wall to lessen cell proliferation. The active agents should then continue to dissolute at different rates and in different phases for up to about one to six months in total.

The invention is not limited by the process used to apply the polymer blends to a substrate surface to form a coating. Examples of suitable coating processes include solution processes, powder coating, melt extrusion, or vapor deposition.

A preferred method is solution coating. For solution coating processes, examples of solution processes include spray coating, dip coating, and spin coating. Typical solvents for use in a solution process include tetrahydrofuran (THF), methanol, ethanol, ethylacetate, dimethylformamide (DMF), dimethyacetamide (DMA), dimethylsulfoxide (DMSO), dioxane, N-methyl pyrollidone, chloroform, hexane, heptane, cylcohexane, toluene, formic acid, acetic acid, and/or dichloromethane. Single coats or multiple thin coats can be applied.

Similarly, the invention is not limited by the process used to form the miscible polymer blends into shaped objects. Such methods would depend on the type of shaped object. Examples of suitable processes include extrusion, molding, micromachining, emulsion polymerization methods, electrospray methods, the reflow method described in Applicants' Assignee's copending U.S. Provisional Application Serial No. 60/403,479, filed on Aug. 13, 2002; U.S. patent application Ser. No. 10/640,701, filed on Aug. 13, 2003; and PCT International Patent Application No. PCT/US 03/25463, filed Aug. 13, 2003 (published as WO 2004/014453A1 on Feb. 19, 2004), all of which are entitled MEDICAL DEVICE EXHIBITING IMPROVED ADHESION BETWEEN POLYMERIC COATING AND SUBSTRATE, etc.

### EXAMPLE

Objects and advantages of this invention are further illustrated by the following example, but the particular materials and amounts thereof recited in this example, as well as other conditions and details, should not be construed to unduly limit this invention.

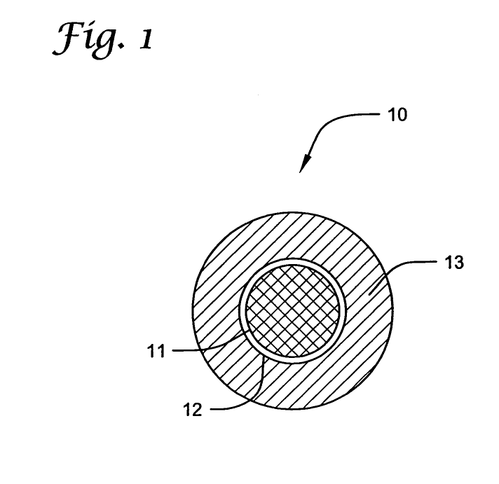

In one example, stainless steel coronary stents (manufactured by Medtronic AVE) were ultrasonically cleaned with isopropanol for about 30 minutes and dried thoroughly prior to spraying with a 0.25% solution of TECOPLAST polyurethane (Thermedics Polymer) in THF as an initial primer. The stents were then heat-treated at 215-220° C. for 5-15 minutes to create better adhesion between metal and polymer interface. Next, each stent was sprayed with 1% solution of mycophenolic acid (Sigma-Aldrich) and sulfasalazine (Sigma-Aldrich) in TECOPLAST polyurethane (30% loading) using THF as solvent. The ratio of mycophenolic acid to sulfasalazine was 1:1. The coating mass of active agents and polymer was approximately 1 milligram (mg), which is corresponding to a coating thickness of about 10 micrometers (μm). The stent was then vacuum-dried in an oven at 45° C. overnight and weighed to determine the theoretical content of active agents. The design of this system 10 is shown in FIG. 1, wherein the stent wire 11 is coated with a primer layer 12, which is coated with a single layer 13 of a TECOPLAST polyurethane with mycophenolic acid and sulfasalazine in a 1:1 ratio. The mycophenolic acid could be present, for example, in an amount of about 5 wt-% to about 20 wt-%. The sulfasalazine could be present, for example, in an amount of about 5 wt-% to about 30 wt-%. The ratio of mycophenolic acid to sulfasalazine could be, for example, within a range of about 1:1 to about 1:5.

The in vitro elution kinetics of dual active agent release was carried out in PBS and at 37° C. The stent was crimped on a stent delivery system and then expanded. After expansion, the physical aspects of the stent were noted prior to placing the stent inside a vial containing 3 milliliters (ml) of PBS. The vial was placed in a shaker at 37° C. and at certain time intervals; the whole solution (3 ml) was removed and replaced with fresh PBS. The amount of each active agent in each dual release system was determined by UV-Vis spectrophotometer using wavelengths of pure active agents at 250 nanometers (nm) for mycophenolic acid and at 359 nm for sulfasalazine, and then solving simultaneous equations of active agent mixtures.

Although this example does not demonstrate an active agent delivery system with a polymer blend, it shows that differential release of two active agents can be achieved by selecting active agents with different molecular weights and solubility parameters. This can also be accomplished by using a polymer blend that matches with the solubility of the faster released active agent.

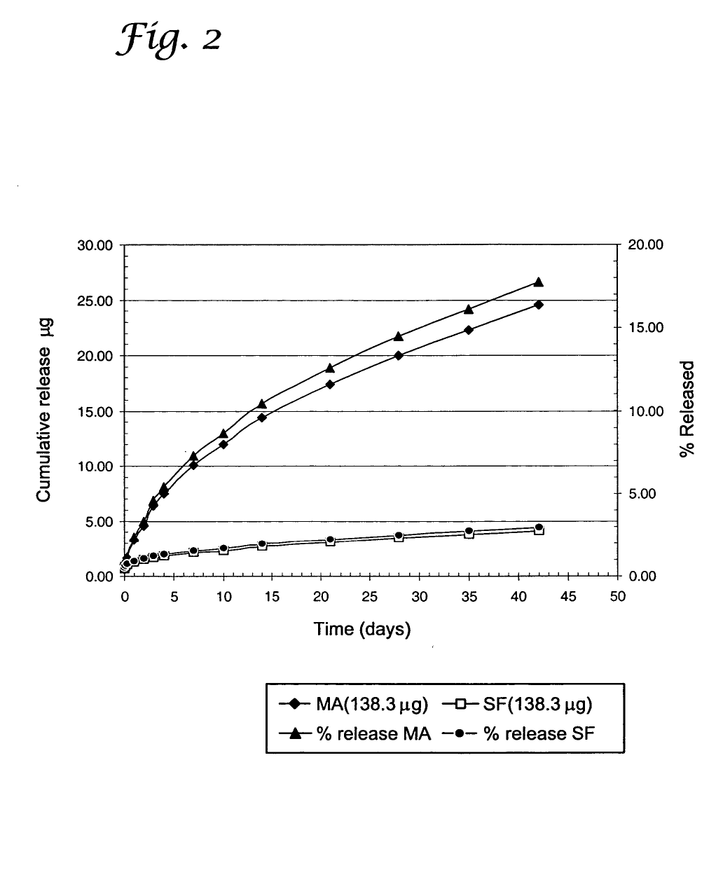

The release characteristics of both active agents are shown in FIG. 2. When two active agents with similar solubility parameters ((23-24 J/cm³)^{0.5}) and with similar loadings in a single polymer, the active agent that has lower molecular weight (in this case mycophenolic acid with a molecular weight of 320) tends to elute faster than sulfasalazine (which has a molecular weight of 398).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. An active agent delivery system comprising two or more active agents in a layer comprising a miscible polymer blend comprising at least two miscible polymers; wherein delivery of at least one of the active agents occurs predominantly under permeation control; and further wherein the permeability of the active agent that is to be released faster is greater than the permeability of the other one or more active agents.

2. The active agent delivery system of claim 1 wherein the difference between the solubility parameter of the active agent that is to be released faster and to be present in a greater amount and the molar average solubility parameter of the at least two miscible polymers is smaller than the differences between the solubility parameter of each of the other one or more active agents and the molar average solubility parameter of the at least two miscible polymers.

3. The system of claim 1 wherein the miscible polymer blend is hydrophilic and comprises a hydrophilic polymer and a second polymer having a different swellability in water at 37° C., wherein the swellability of the miscible polymer blend controls the delivery of the active agents.

4. The system of claim 3 wherein the hydrophilic polymer is a hydrophilic polyurethane.

5. The system of claim 3 wherein the hydrophilic polymer is selected from the group consisting of polyvinyl pyrrolidone, polyvinyl alcohol, polypropylene oxide, polyethylene oxide, polystyrene sulfonate, polysaccharide, and combinations thereof.

6. The system of claim 3 wherein the miscible polymer blend comprises a polyvinyl pyrollidone-co-vinyl acetate copolymer and a poly(ether urethane).

7. The system of claim 3 wherein the second polymer is a hydrophilic polymer or a hydrophobic polymer.

8. The system of claim 7 wherein the second polymer is a hydrophilic polyurethane.

9. The system of claim 8 wherein the hydrophilic polyurethane comprises soft segments comprising polyethylene oxide units.

10. The system of claim 1 wherein the miscible polymer blend comprises a polyurethane and a second polymer.

11. The system of claim 10 wherein the second polymer has at least one Tg equal to or higher than all Tg's of the polyurethane.

12. The system of claim 10 wherein the active agent is not heparin.

13. The system of claim 10 wherein the second polymer is selected from the group consisting of a polycarbonate, a polysulfone, a polyurethane, a polyphenylene oxide, a polyimide, a polyamide, a polyester, a polyether, a polyketone, a polyepoxide, a styrene-acrylonitrile copolymer, and combinations thereof.

14. The system of claim 10 wherein the polyurethane has a Shore durometer hardness of about 70D to about 80D.

15. The system of claim 10 wherein the second polymer is a polyurethane having a Shore durometer hardness of about 80D to about 90D.

16. The system of claim 10 wherein the second polymer is a polycarbonate.

17. The system of claim 10 wherein the polyurethane is a poly(carbonate urethane) or a poly(ether urethane).

18. The system of claim 10 wherein:
each active agent has a solubility parameter, the polyurethane has a soft segment solubility parameter and a hard segment solubility parameter, and the second polymer has at least one solubility parameter; and
at least one of the following relationships is true:
the difference between the solubility parameter of each active agent and the solubility parameter of the polyurethane hard segment is no greater than about 10 J^{1/2}/cm^{3/2};
the difference between the solubility parameter of each active agent and the solubility parameter of the polyurethane soft segment is no greater than about 10 J^{1/2}/cm^{3/2}; and
the difference between the solubility parameter of each active agent and at least one solubility parameter of the second polymer is no greater than about 10 J^{1/2}/cm^{3/2}.

19. The system of claim 10 wherein:
the polyurethane has a soft segment solubility parameter and a hard segment solubility parameter, and the second polymer has at least one solubility parameter; and
at least one of the following relationships is true:
the difference between the solubility parameter of the polyurethane hard segment and at least one solubility parameter of the second polymer is no greater than about 5 J^{1/2}/cm^{3/2}; and
the difference between the solubility parameter of the polyurethane soft segment and at least one solubility parameter of the second polymer is no greater than about 5 J^{1/2}/cm^{3/2}.

20. The system of claim 1 wherein the miscible polymer blend comprises a hydrophobic cellulose derivative and a polyvinyl homopolymer or copolymer selected from the group consisting of a polyvinyl alkylate homopolymer or copolymer, a polyvinyl alkyl ether homopolymer or copolymer, a polyvinyl acetal homopolymer or copolymer, and combinations thereof.

21. The system of claim 20 wherein:
each of the active agents, the hydrophobic cellulose derivative, and the polyvinyl homopolymer or copolymer has a solubility parameter; and
at least one of the following relationships is true:
the difference between the solubility parameter of each active agent and the solubility parameter of the hydrophobic cellulose derivative is no greater than about 10 J^{1/2}/cm^{3/2}; and
the difference between the solubility parameter of each active agent and at least one solubility parameter of the polyvinyl homopolymer or copolymer is no greater than about 10 J^{1/2}/cm^{3/2}.

22. The system of claim 20 wherein each active agent has a solubility parameter within at least about 10 J^{1/2}/cm^{3/2} of the solubility parameters of each of cellulose acetate butyrate and polyvinyl acetate.

23. The system of claim 20 wherein:
each of the hydrophobic cellulose derivative and the polyvinyl homopolymer or copolymer has a solubility parameter; and
the difference between the solubility parameter of the hydrophobic cellulose derivative and at least one solubility parameter of the polyvinyl homopolymer or copolymer is no greater than about 5 J^{1/2}/cm^{3/2}.

24. The system of claim 20 wherein the hydrophobic cellulose derivative is selected from the group consisting of methyl cellulose, ethyl cellulose, hydroxy propyl cellulose, cellulose acetate, cellulose propionate, cellulose butyrate, cellulose nitrate, and combinations thereof.

25. The system of claim 20 wherein the polyvinyl homopolymer or copolymer is a polyvinyl alkylate homopolymer or copolymer.

26. The system of claim 25 wherein the polyvinyl alkylate homopolymer or copolymer is a homopolymer or copolymer of polyvinyl acetate, polyvinyl propionate, or polyvinyl butyrate.

27. The system of claim 25 wherein the polyvinyl alkylate homopolymer or copolymer is a polyvinyl acetate homopolymer or copolymer.

28. The system of claim 1 wherein the miscible polymer blend comprises a poly(ethylene-co-(meth)acrylate) and a second polymer not including poly(ethylene vinyl acetate).

29. The system of claim 28 wherein:
each of the active agents, the poly(ethylene-co-(meth)acrylate) and the second polymer has a solubility parameter; and
at least one of the following relationships is true:
the difference between the solubility parameter of each active agent and the solubility parameter of the poly(ethylene-co-(meth)acrylate) is no greater than about 10 J^{1/2}/cm^{3/2}; and
the difference between the solubility parameter of each active agent and at least one solubility parameter of the second polymer is no greater than about 10 J^{1/2}/cm^{3/2}.

30. The system of claim 28 wherein:
each of the poly(ethylene-co-(meth)acrylate) and the second polymer has a solubility parameter; and
the difference between the solubility parameter of the poly(ethylene-co-(meth)acrylate) and at least one solubility parameter of the second polymer is no greater than about 5 J^{1/2}/cm^{3/2}.

31. The system of claim 28 wherein the second polymer is a polyvinyl alkylate homopolymer or copolymer.

32. The system of claim 28 wherein the second polymer is a polyalkyl and/or aryl methacrylate or acrylate or copolymer.

33. The system of claim 28 wherein the second polymer is a polyvinyl acetal or copolymer.

34. The system of claim 1 wherein the miscible polymer blend comprises a copolymer of a methacrylate, a vinyl acetate, and a vinyl pyrrolidone.

35. The system of claim 1 wherein a first active agent is selected from the group consisting of indomethacin, sulindac, diclofenal, etodolac, meclofenate, mefenamic acid, nambunetone, piroxicam, phenylgutazone, meloxicam, dexamethoasone, betamethasone, dipropionate, diflorsasone diacetate, clobetasol propionate, galobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin, curcumin, a proteasome inhibitor, antisense c-myc, celocoxib, valdecoxib, and combinations thereof.

36. The system of claim 35 wherein a second active agent is released at a slower rate than that of the first active agent, after the start of release of the first active agent, or both.

37. The system of claim 36 wherein the second active agent is selected from the group consisting of podophyllotoxin, mycophenolic acid, teniposide, etoposide, trans-retinoic acids, 9-cis retinoic acid, 13-cis retinoic acid, rapamycin, a rapalog, camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof.

38. The system of claim 1 wherein at least one active agent is selected from the group consisting of podophyllotoxin, mycophenolic acid, teniposide, etoposide, camptothecin, irinotecan, topotecan, mithramycin, and combinations thereof.

39. The system of claim 38 further wherein one active agent is sulfasalzine.

40. The system of claim 38 further wherein one active agent is indomethacin.

41. The system of claim 38 further wherein one active agent is ascomycin.

42. The system of claim 38 further wherein one active agent is leflunomide.

43. The system of claim 38 further wherein one active agent is dexamethasone.

44. The system of claim 38 further wherein one active agent is piroxicam.

45. The system of claim 38 further wherein one active agent is beclomethasone dipropionate.

46. The system of claim 38 further wherein one active agent is S-nitrosoglutathione.

47. The system of claim 1 wherein at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

48. The system of claim 47 further wherein-one active agent is rosiglitazone.

49. The system of claim 47 further wherein one active agent is troglitazone.

50. The system of claim 47 further wherein one active agent is pioglitazone.

51. A medical device comprising the active agent delivery system of claim 1.

52. The medical device of claim 51 selected from the group consisting of a stent, stent graft, anastomotic connector, lead, needle, guide wire, catheter, sensor, surgical instrument, angioplasty balloon, wound drain, shunt, tubing, urethral insert, pellet, implant, blood oxygenator, pump, vascular graft, valve, pacemaker, orthopedic device, replacement device for nucleus pulposus, and intraocular lense.

53. A stent comprising the active agent delivery system of claim 1.

54. A medical device comprising:
a substrate surface;
a polymeric undercoat layer adhered to the substrate surface; and
an active agent delivery system adhered to the polymeric undercoat layer;
wherein the active agent delivery system comprises two or more active agents in a layer comprising a miscible polymer blend comprising at least two miscible polymers; wherein delivery of at least one of the active agents occurs predominantly under permeation control; and further wherein the permeability of the active agent that is to be released faster is greater than the permeability of the other one or more active agents.

55. The medical device of claim 54 wherein the difference between the solubility parameter of the active agent that is to be released faster and to be present in a greater amount and the molar average solubility parameter of the at least two miscible polymers is smaller than the differences between the solubility parameter of each of the other one or more active agents and the molar average solubility parameter of the at least two miscible polymers.

56. A stent comprising:
a substrate surface;
a polymeric undercoat layer adhered to the substrate surface; and
an active agent delivery system adhered to the polymeric undercoat layer;
wherein the active agent delivery system comprises two or more active agents in a layer comprising a miscible polymer blend comprising at least two miscible polymers; wherein delivery of at least one of the active agents occurs predominantly under permeation control; and further wherein the permeability of the active agent that is to be released faster is greater than the permeability of the other one or more active agents.

57. A method of designing an active agent delivery system for delivering two or more active agents over a preselected dissolution time (t) through a preselected critical dimension (x) of a miscible polymer blend, the method comprising:
providing two or more active agents having a molecular weight no greater than about 1200 g/mol;
selecting at least two miscible polymers to form the miscible polymer blend, wherein:
the permeability of the active agent that is to be released faster is greater than the permeability of the other one or more active agents;
the difference between the solubility parameter of each active agent and the molar average solubility parameter of the at least two miscible polymers is no greater than about 10 J^{1/2}/cm^{3/2};
the difference between at least one solubility parameter of each of the at least two miscible polymers is no greater than about 5 J^{1/2}/cm^{3/2};
the difference between the solubility parameter of the active agent that is to be released faster and in a greater amount and the molar average solubility parameter of the at least two miscible polymers is smaller than the differences between the solubility parameter of each of the other one or more active agents and the molar average solubility parameter of the at least two miscible polymers; and
the difference between at least one Tg of each of the at least two polymers is sufficient to include the target diffusivity;
combining the at least two miscible polymers to form a miscible polymer blend;
and
combining the miscible polymer blend with the active agents to form an active agent delivery system having the preselected dissolution time through a preselected critical dimension of the miscible polymer blend, wherein delivery of at least one of the active agents occurs predominantly under permeation control.

58. A method of designing an active agent delivery system for delivering two or more active agents over a preselected dissolution time (t) through a preselected critical dimension (x) of a miscible polymer blend, the method comprising:
providing two or more active agents having a molecular weight greater than about 1200 g/mol;
selecting at least two miscible polymers to form the miscible polymer blend, wherein:
the permeability of the active agent that is to be released faster is greater than the permeability of the other one or more active agents;
the difference between the solubility parameter of each active agent and the molar average solubility parameter of the at least two miscible polymers is no greater than about 10 J^{1/2}/cm^{3/2};
the difference between at least one solubility parameter of each of the at least two miscible polymers is no greater than about 5 J^{1/2}/cm^{3/2};
the difference between the solubility parameter of the active agent that is to be released faster and in a greater amount and the molar average solubility parameter of the at least two miscible polymers is smaller than the differences between the solubility parameter of each of the other one or more active agents and the molar average solubility parameter of the at least two miscible polymers; and
the difference between the swellabilities of the at least two miscible polymers is sufficient to include the target diffusivity;
combining the at least two miscible polymers to form a miscible polymer blend;
and
combining the miscible polymer blend with the active agents to form an active agent delivery system having the preselected dissolution time through a preselected critical dimension of the miscible polymer blend, wherein delivery of at least one of the active agents occurs predominantly under permeation control.

59. A method for delivering two or more active agents to a subject, the method comprising:
providing an active agent delivery system of claim 1; and
contacting the active agent delivery system with a bodily fluid, organ, or tissue of a subject.

60. A method for delivering two or more active agents to a subject, the method comprising:
providing an active agent delivery system of claim 2; and
contacting the active agent delivery system with a bodily fluid, organ, or tissue of a subject.

61. A method for delivering two or more active agents to a subject, the method comprising:
providing an active agent delivery system of claim 3; and
contacting the active agent delivery system with a bodily fluid, organ, or tissue of a subject.

62. A method for delivering two or more active agents to a subject, the method comprising:
providing an active agent delivery system of claim 10; and
contacting the active agent delivery system with a bodily fluid, organ, or tissue of a subject.
